(19) United States
(12) Patent Application Publication
(10) Pub. No.: US 2010/0034762 A1
(43) Pub. Date: Feb. 11, 2010

(54) Novel enrichment methods for gallic acid esters including 1-O-galloy1-bata-D-glucose and mucic acid gallates medicaments, therapeutic applications and methods of treatment thereof

(76) Inventors: Muhammed Majeed, Piscataway, NJ (US); Atul Namdevrao Jadhav, Bangalore (IN); Jyotish Shanker Srivastava, Bangalore (IN); Subbalakshmi Prakash, Piscataway, NJ (US); Kalyanam Nagabhushanam, Piscataway, NJ (US); Beena Bhat, Bangalore (IN)

Correspondence Address: SABINSA CORPORATION, 70 ETHEL ROAD WEST, UNIT 6, PISCATAWAY, NJ, 08854, US

(21) Appl. No.: 12/208,024

(22) Filed: Sep. 10, 2008

Related U.S. Application Data
(60) Provisional application No. 61/086,501, filed on Aug. 6, 2008.

(51) Int. Cl. A61Q 17/04; C07H 17/02; C07C 69/76; C07C 63/04; C07D 311/78; A61P 35/00; A61K 31/7088; A61K 31/235; A61K 31/19; A61K 31/35

(52) U.S. Cl. 424/60; 536/18.5; 560/103; 562/493; 549/384; 514/32; 514/532; 514/568; 514/453; 424/59

(57) ABSTRACT

Improved methods to prepare aqueous extracts of gallic acid esters including 1-O-galloyl-β-D-glucose (β-glucogallin) and other mucic acid gallates from the fruits of Emblica officinalis, wherein enrichment of gallic acid esters is achieved by Supercritical Fluid Extraction (SCFE) procedure, are disclosed. The use of such standardized extracts of gallic acid esters (greater than 40% w/w of gallic acid esters) in the manufacture of medicaments with diverse health benefits, is also disclosed.

This application is a non-provisional filing of provisional U.S. application No. 61/086,501 filed on Aug. 6, 2008.

### BACKGROUND OF THE INVENTION

The invention in general relates to improvements in the methods of extracting the enriched gallic esters including 1-O-galloyl-β-D-glucose and mucic acid gallates from the fruits of Emblica officinalis. In particular, the present invention relates to improvements in the art for the aqueous extraction of gallic acid esters including 1-O-galloyl-β-D-glucose and mucic acid gallates from the fruits of Emblica officinalis, where the improvement involves enrichment of gallic acid esters by employing Super Critical Fluid Extraction (SCFE) procedure after the steps of aqueous extraction, SCFE selectively removing medium polar constituents of the aqueous extract including gallic acid and ellagic acid to result in a standardized Emblica officinalis extract enriched in the said gallic acid esters. In an alternate embodiment, the invention also discloses improvements in the art of extracting gallic acid esters from the fruits of Emblica officinalis using Super Critical Fluid Extraction (SCFE) as the principal extraction process to obtain standardized enriched gallic acid esters by overcoming the disadvantages of conventional extraction methods where gallic acid esters are hydrolysed to gallic acid resulting in low levels of desired gallic acid esters. Further disclosed are medicaments, therapeutic applications and methods of treatment employing gallic acid esters enriched Emblica officinalis extracts (greater than 40% w/w gallic acid esters) obtained from the aforesaid improvements.

### DESCRIPTION OF PRIOR ART

Enriched constituents extracted from the fruits of Emblica officinalis have been reported in numerous prior art references. Some important references have been included herein below.
A. Process for obtaining a stabilized antioxidant formulation from Emblica officinalis fruit comprising extracting the finely pulped fruit with a dilute aqueous or alcoholicwater salt solution at a temperature of about 70° C. 5° C. to form an extractcontaining solution, filtering, and drying to provide the desired antioxidant blend as a powder has been discussed in WO/2000/048551, U.S. Pat. No. 6,124,268, U.S. Pat. No. 6,235,721 to Ghosal, Shibnath. The main constituents from the pericarp of Emblica officinalis discussed here include
1. Emblicanin-A: 2,3-di-O-galloyl-4,6-(S)-hexahydroxydiphenoyl-2-keto-glucono-lactone;
2. Emblicanin-B: 2,3,4,6-bis-(S)-hexahydroxydiphenoyl-2-keto-glucono-lactone;
3. Punigluconin: 2,3-di-O-galloyl4,6-(S)-hexahydroxydiphenoyl gluconic acid;
4. Pedunculagin: 2,3,4,6-bis-(S)-hexahydroxydiphenoyl-D-glucose;
5. Rutin: 3′,4′,5,7-tetrahydroxyflavono-1,3-O-rhamnoglucoside;
6. Gallo-ellagitannoids; 7. Gallic acid and 8. Ellagic acid.
B. Sun protective and skin lightening dosage forms of Emblica officinalis extract comprising Emblicanin A, Emblicanin B, Pendunculagin and Punigluconin, preferably in an amount of >40% by weight of the extract have been discussed in US20050089590, EP1560561, US20030198612 and US20040086560.
C. Mucic acid 2-O-gallate, mucic acid 1,4-lactone 2-O-gallate and mucic acid 1,4-lactone 5-O-gallate were identified as the principal phenolic constituents of Emblica officinalis juice by Zhang Y J, Tanaka T, Yang C R, Kouno I (Chem Pharm Bull (Tokyo). May 2001;49(5):537-40.) Occurring with 1-O-galloyl-β-D-glucose, the probability of these galloyl esters having anti-oxidant activity along with vitamin C (ascorbic acid) has been indicated although no proof of evidence has been given to substantiate this claim.
D. With considerable doubts on the quantitative ability and role of ascorbic acid in potentiating the therapeutic effects of Emblica officinalis [I. “A much higher concentration of Ascorbic acid is required to effect the same inhibition as Amla—S. M. Khopde et al., Current Science, Vol. 81, No. 2, 25 Jul. 2001; and II. S. Ghosal et al., Indian J of Chemistry, Vol. 35B, 1996, pp 941-948], the present inventors sought to revisit the chemistry behind amla extracts in detail to unravel unknown data if any and if required correct popularly existing misconceptions.

Chemical Analysis of Emblica officinalis by the Present Inventors:

The present inventors separated the spray dried extract of Emblica officinalis into seven major fractions using Preparative Shimadzu HPLC system equipped with Class Vp software with binary gradient (LC8A) pump, UV-VIS dual wavelength detector (SPD-10AVp), system controller (SCL-10AVp) and Rheodyne injector 7725i with 5 mL sample loop. Separation was done on Phenomenex Jupiter C18 column 300A(250×50 mm, 15 [symbol] ) with mobile phase A-water (0.1% formic acid) and B-methanol. A gradient was used as 0-35 min 5% B, 35-40 min 100% B, 40-50 min. 100% B, 50-51 min. 5% B and 51-60 min. 5% B. Flow rate was 35 ml/min and monitoring at 240 and 280 nm.

Based on extensive chemical analysis of the peaks obtained in the preparative HPLC profile, the present inventors concluded the absence of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

Further, the present inventors also concluded that 1-O-galloyl-β-D-glucose along with mucic acid gallates were the principal components of Emblica officinalis fruit.

Still further, the present inventors attempted methods to disclose important biological properties of such enriched extracts which would probably represent the actual therapeutic potential of Emblica officinalis.

With the confirmation of 1-O-galloyl-β-D-glucose and other mucic acid gallates as the principal components of Emblica officinalis, the present inventors sought to overcome the difficulties associated with very low levels of isolation of these compounds reported earlier[Chem Pharm Bull (Tokyo). May 2001;49(5):537-40].

It is the principal object of the present invention to devise improvements in processes known in the art for obtaining standardized Emblica officinalis extracts enriched in 1-O-galloyl-β-D-glucose and other mucic acid gallates.

It is another object of the present invention to study the biological properties of standardized Emblica officinalis extracts enriched in 1-O-galloyl-β-D-glucose and other mucic acid gallates, propose their use in the manufacture of medicaments for appropriate medical conditions and in suitable therapeutic formulations.

Further, the invention also aims to disclose treatment methods using appropriate dosage regimens of standardized extracts of Emblica officinalis enriched in 1-O-galloyl-β-D-glucose and other mucic acid gallates obtained through improvements disclosed in the current invention.

The present invention fulfills the aforesaid objectives and provides further related advantages.

### SUMMARY OF THE INVENTION

The present invention relates to improvements in the art for the aqueous extraction of gallic acid esters including 1-O-galloyl-β-D-glucose and other mucic acid gallates from the fruits of Emblica officinalis, where the improvement involves enrichment of gallic acid esters by employing Super Critical Fluid Extraction (SCFE) procedure after the steps of aqueous extraction, SCFE selectively removing gallic acid, ellagic acid and other medium polar constituents of the aqueous extract to result in a standardized extract enriched in the said gallic acid esters. In an alternate embodiment, the invention also discloses improvements in the art of extracting gallic acid esters from the fruits of Emblica officinalis using Super Critical Fluid Extraction (SCFE) as the principal extraction process to obtain standardized enriched gallic acid esters (greater than 40% w/w of gallic acid esters) by overcoming the disadvantages of conventional extraction methods where hydrolysis of the gallic acid esters to gallic acid yield low levels of gallic acid esters. The standardized extracts enriched in gallic acid esters obtained through the aforesaid improvements occur completely devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) ascorbic acid. The present invention also discloses the use of the standardized extracts of gallic acid esters (greater than 40% w/w of gallic acid esters) obtained through the improvements of the present invention in manufacture of medicaments
(i) that have therapeutic potential as anti-oxidants;
(ii) for the prevention of serum LDL oxidation;
(iii) for the treatment of androgenic alopecia;
(iv) for the treatment of chronic abacterial prostatitis, prostatic hyperplasia and prostatic cancer;
(v) For the photo protection to UVA and UVB rays;
(vi) For the treatment of diabetes mellitus; and
(vii) For the treatment of diabetic retinopathy.
   Also disclosed are methods of
I. Preventing anti-oxidant damage in mammals; (ii) Therapeutically reducing serum LDL oxidation; (iii) treating androgenic alopecia; and (iv) treating chronic abacterial prostatitis, prostatic hyperplasia and prostatic cancer; (v) treating diabetes mellitus; and (vi) treating diabetic retinopathy using orally administered medicaments comprising an effective amount of 400 mg/day of standardized extracts enriched in gallic acid esters (greater than 40% w/w of gallic acid esters) obtained through the improvements of the present invention.
II. Enhancing photo protection to UVA and UVB rays using topically applied medicaments that comprise of standardized Emblica officinalis extracts enriched in gallic acid esters (greater than 40% w/w of gallic acid esters) obtained through the improvements of the present invention to exposed areas of skin that face the risk of UV A and UV B induced photo damage.

### BRIEF DESCRIPTION OF THE DRAWINGS

FIG. 1 shows the preparative HPLC profile of the spray dried extract of Emblica officinalis elucidating the chemistry of amla as proposed by the present inventors.

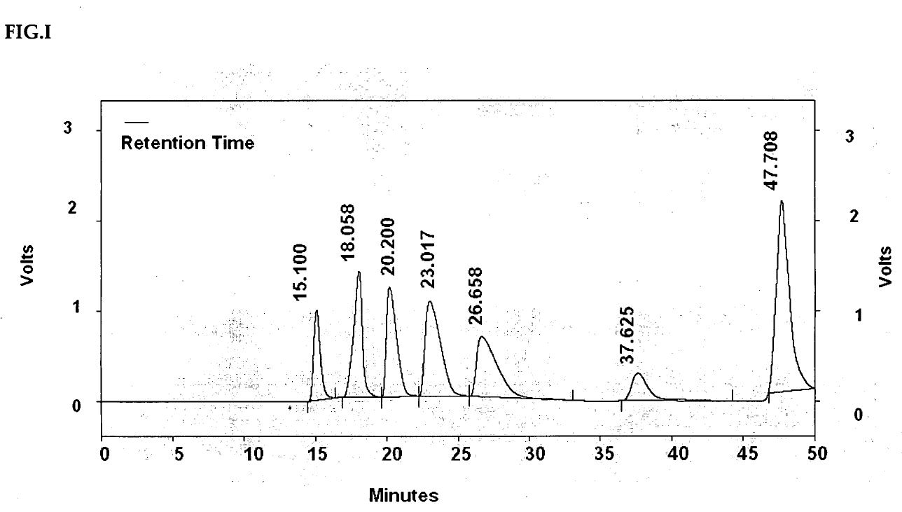

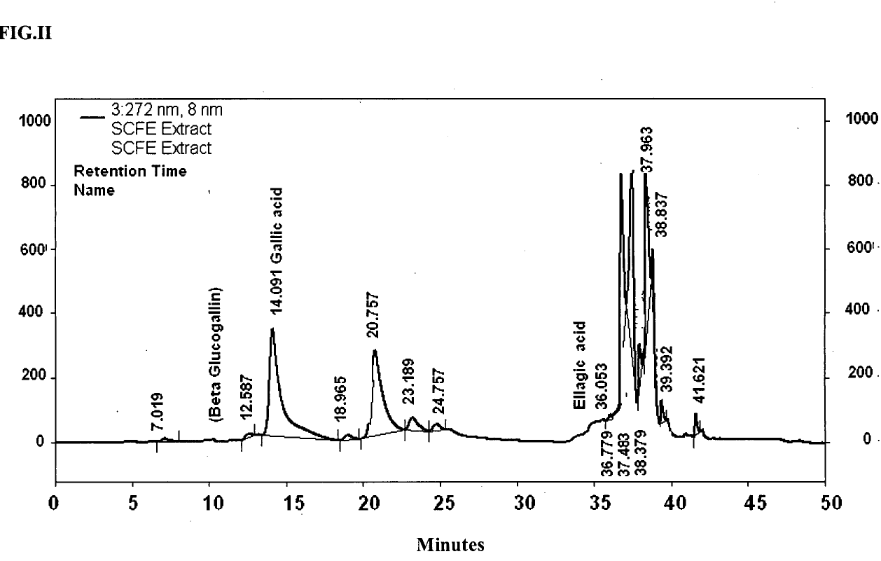

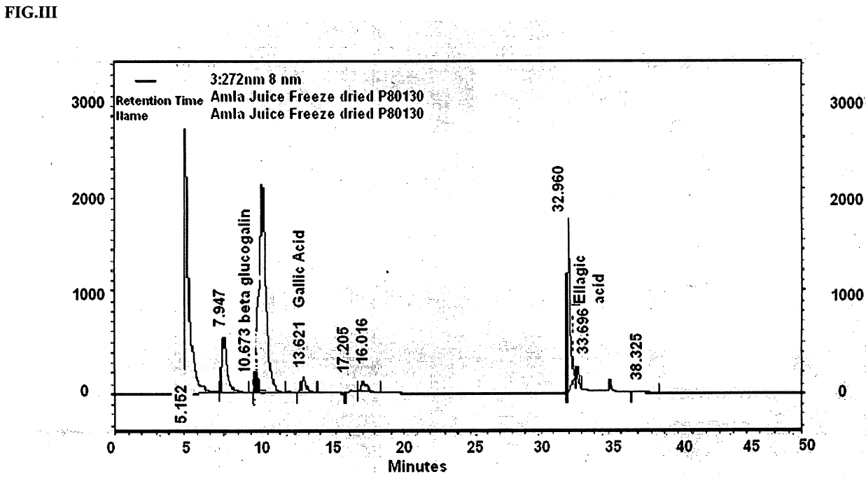

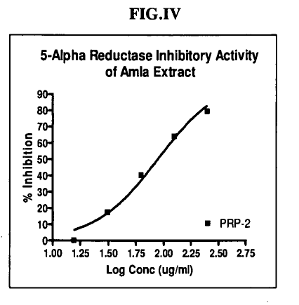

FIG. II shows the HPLC chromatogram depicting the extract obtained when SCFE is applied after aqueous extraction of the fruits of Emblica officinalis. The chromatogram clearly evinces the removal of gallic acid, ellagic acid and other medium polar constituents from the extract leading to an extract enriched in gallic acid esters alone.

FIG. III shows the HPLC chromatogram depicting the SCFE extracts of amla where the content of gallic acid esters is greater than 40% w/w than the usual 7% w/w obtained through aqueous extraction of the fruits. SCFE avoids the disadvantages of the hydrolysis of susceptible gallic acid esters to yield an extract enriched in gallic acid esters.

FIG. IV is a graphical representation of the 5-alpha reductase activity [% Inhibition] of the standardized extract of amla containing >40% w/w of gallic acid esters including 1-O-galloyl-β-D-glucose and mucic acid gallates obtainined by the improvements of the present invention.

### DESCRIPTION OF THE MOST PREFERRED EMBODIMENT

In the most preferred embodiment, the present invention relates to improvements in the art for the aqueous extraction of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 from the fruits of Emblica officinalis, said improvement in the method of enriching said gallic acid esters by including Super Critical Fluid Extraction (SCFE) process after the steps of the aqueous extraction, SCFE selectively removing medium polar constituents including gallic acid represented by STR#6 and ellagic acid represented by STR#7 from the aqueous extract to result in a standardized Emblica officinalis extract enriched in the said gallic acid esters. More specifically, the said standardized extract comprises greater than 40% w/w gallic acid esters. Still more specifically, the said standardized extract is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

In an alternate embodiment, the present invention relates to improvements in the art for the extraction of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 from the fruits of Emblica officinalis, the improvement consisting essentially the use of Super Critical Fluid Extraction (SCFE) process as the principal extraction process to obtain standardized Emblica officinalis extract enriched in gallic acid esters by overcoming the disadvantages of conventional extraction methods where hydrolysis of the gallic acid esters to gallic acid represented by STR#6 yield low levels of gallic acid esters. More specifically, the said standardized extract comprises greater than 40% w/w of gallic acid esters. Still more specifically, the said standardized extract is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. In preferred embodiments the entrant is acetone or an alcohol selected from the group consisting of aliphatic and aromatic alcohols.

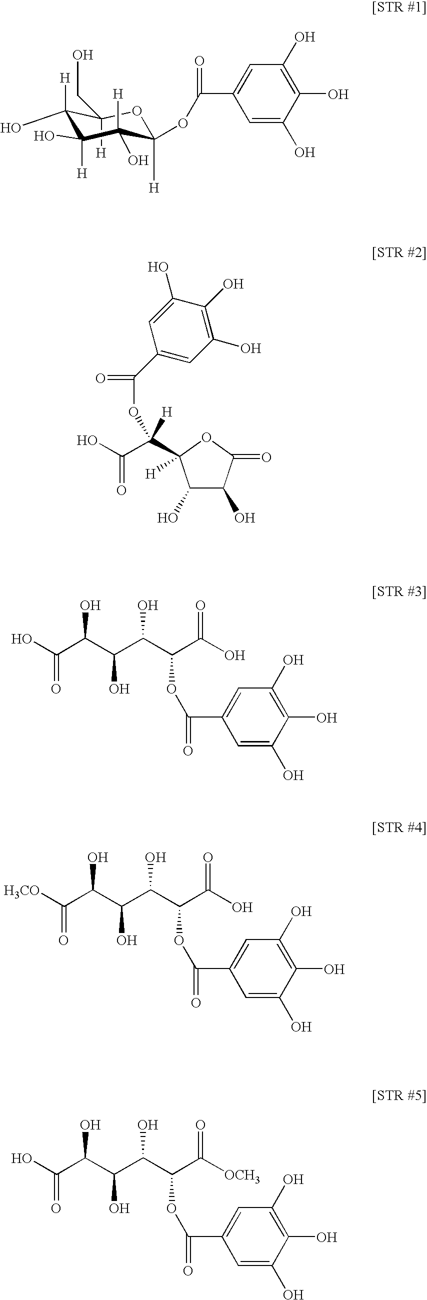

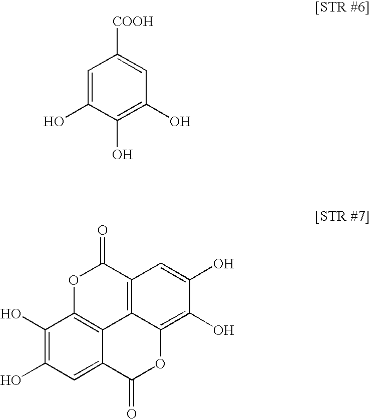

In another preferred embodiment, the present invention relates to the use of standardized Emblica officinalis extracts enriched in greater than 40% w/w of gallic acid esters through the improvements of the present invention in the manufacture of medicaments for the treatment of androgenic alopecia.

In another preferred embodiment, the present invention relates to the use of standardized Emblica officinalis extracts enriched in greater than 40% w/w of gallic acid esters through the improvements of the present invention in the manufacture of medicaments for the treatment of chronic abacterial prostatitis/chronic pelvic pain syndrome.

In another preferred embodiment, the present invention relates to the use of standardized Emblica officinalis extracts enriched in greater than 40% w/w of gallic acid esters through the improvements of the present invention in the manufacture of medicaments for the treatment of benign prostatic hyperplasia.

In another preferred embodiment, the present invention relates to the use of standardized Emblica officinalis extracts enriched in greater than 40% w/w of gallic acid esters through the improvements of the present invention in the manufacture of medicaments for the treatment of prostatic cancer. The present invention includes other forms of cancer as well.

In another preferred embodiment, the present invention relates to the use of standardized Emblica officinalis extracts enriched in greater than 40% w/w of gallic acid esters through the improvements of the present invention in the manufacture of medicaments for skin protection against UVA and UVB rays.

In another preferred embodiment, the present invention relates to the use of standardized Emblica officinalis extracts enriched in greater than 40% w/w of gallic acid esters through the improvements of the present invention in the manufacture of medicaments that would retard serum low-density lipoprotein (LDL) or Very low-density lipoprotein (VLDL) oxidation.

In another preferred embodiment, the present invention relates to the use of standardized Emblica officinalis extracts enriched in greater than 40% w/w of gallic acid esters through the improvements of the present invention in the manufacture of anti-oxidant medicaments that would protect the body systems from oxidative damage caused by free radicals.

In another preferred embodiment, the present invention relates to the use of standardized Emblica officinalis extracts enriched in greater than 40% w/w of gallic acid esters through the improvements of the present invention in the manufacture of medicaments for the treatment of type II diabetes mellitus.

In another preferred embodiment, the present invention relates to the use of standardized Emblica officinalis extracts enriched in greater than 40% w/w of gallic acid esters through the improvements of the present invention in the manufacture of medicaments for the treatment of diabetic retinopathy.

In yet another preferred embodiment, the present invention also relates to method of treatment using medicaments comprising an effective amount of 400 mg/day of standardized Emblica officinalis extracts enriched in greater than 40% w/w of gallic acid esters through the improvements of the present invention, wherein said medicaments are administered orally in patients who are in need of treatment for conditions including andrgenic alopecia, chronic abacterial prostatitis, chronic pelvic pain syndrome, benign prostatic hyperplasia, prostatic and other forms of cancer, diabetic retinopathy, type II diabetes mellitus, serum LDL and VLDL oxidation and UVA/UVB damage.

In yet another preferred embodiment, the present invention also relates to methods of treatment using topical medicaments comprising standardized Emblica officinalis extracts enriched in greater than 40% w/w of gallic acid esters through the improvements of the present invention are applied in exposed areas of skin at a risk for UVA and UVB damage.

In further preferred embodiments, the present invention also discloses pharmaceutical, nutraceutical and cosmetic compositions made using standardized Emblica officinalis extracts enriched in greater than 40% w/w of gallic acid esters through the improvements of the present invention, in which said extracts are devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose;
   and
5. Ascorbic acid.

The improvements of the present invention discussed herein above relating to the production of standardized Emblica officinalis extracts enriched in greater than 40% w/w of gallic esters and being devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose;
   and
5. Ascorbic acid, and biological properties there of are discussed herein below as examples.

### EXAMPLE I A

(FIG. II) shows the HPLC chromatogram depicting the extract obtained when SCFE is applied after aqueous extraction of the fruits of Emblica officinalis. The chromatogram clearly evinces the removal of gallic acid, ellagic acid and other medium polar constituents from the extract leading to an extract enriched in gallic acid esters alone.

### EXAMPLE I B

(FIG. III) shows the HPLC chromatogram depicting the direct SCFE extracts of amla where the content of gallic acid esters is greater than 40% w/w than the usual 7% w/w obtained through aqueous extraction of the fruits. SCFE avoids the disadvantages of the hydrolysis of susceptible gallic acid esters to yield an extract enriched in gallic acid esters.

### EXAMPLE IC

Adoption of the AOAC official method 955.35 to estimate the content of gallic acid esters including 1-O-galloyl-β-D-glucose and other mucic acid gallates in extracts of Emblica officinalis obtained through the improvements of the present invention, resulted in the percentage concentration of gallic acid esters (total tannins) from 40%-100% on a continuous basis in different batches of extracts.

### EXAMPLE II

### (Table A)—Anti-Oxidant Activity and Anti-Cholesteremic Activity

Though the anti-oxidant activity of very low levels of detected of gallic acid esters along with Vitamin C in a 60% aqueous acetone extract of the powdered form of fruit juice of P. emblica had been suggested in Chem. Pharm. Bull. 49(5) 537-540 (2001), concrete data is still not available. With the present inventors detecting the absence of Vitamin C in amla fruits, such presumptions required to be corroborated. The standardized Emblica officinalis extracts enriched in greater than 40% w/w of gallic acid esters obtained through the improvements involving Supercritical Carbon dioxide extraction process in the present invention show exemplary anti-oxidant effects. Further, the inventors have clearly shown that almost all of the anti-oxidant activity of Emblica officinalis is to be attributed to gallic acid esters including 1-O-galloyl-β-D-glucose and other mucic acid gallates discussed above and not previously reported 1. 2,3-di-O-galloyl-4,6-(S)-hexahydroxydiphenoyl-2-keto-glucono-lactone; 2. 2,3,4,6-bis-(S)-hexahydroxydiphenoyl-2-keto-glucono-lactone; 3. 2,3-di-O-galloyl4,6-(S)-hexahydroxydiphenoyl gluconic acid; 4. 2,3,4,6-bis-(S)-hexahydroxydiphenoyl-D-glucose; and 5. Ascorbic acid, which have been proved to be non-existent. Thus Emblica officinalis extracts enriched in greater than 40% w/w gallic acid esters through the improvements of the present invention qualify not only as novel anti-oxidant formulations but also evince potential of these extracts in other biological properties such as anti-cholesteremic activity. The standard procedures for determining anti-oxidant properties and the results of such assays for the standardized Emblica officinalis extracts enriched in greater than 40% w/w gallic acid esters through the improvements of the present invention are elucidated herein below.
(i) Oxygen Radical Absorbance Capacity (ORAC)—ORAC value is a measure of inhibition of peroxyl radicals. ORAC value is represented as Trolox equivalents where the product is compared with Trolox, a standard inhibitor of peroxyl radicals. Higher the ORAC value, higher is the peroxyl radical inhibition. ORAC value is represented as Trolox equivalents per gm or liter by calculating the area under the curve formed by the inhibition of quenching of fluroscein dye induced by the peroxyl radical generator, azobis.
(ii) DPPH scavenging—Generation of free radicals in skin due to various stress conditions such as UV exposure, pollution, ageing etc, result in induction of melanin synthesis. The quenching of the stable free radical Diphenylpicryl hydrazyl (DPPH) in a methanolic reaction mixture by the antioxidant is an indication of the antioxidant potential.
(iii) Inhibition of Cholesteryl ester transfer protein (CETP) The plasma lipoproteins are continuously remodeled during their transit through the plasma compartment, owing to the action of lipid metabolizing enzymes and lipid transfer process. These activities have a major effect on the composition, size, and concentration of the lipoproteins. Lipid transfer process involving neutral lipids are mediated by specialized plasma proteins, called lipid transfer proteins. The plasma lipid transfer protein includes cholesteryl ester transfer protein (CETP) with specificity for both neutral lipids and phospholipids. CETP is a hydrophilic glycoprotein which is secreted mainly from liver and circulates in plasma mainly bound to high density Lipoprotein (HDL). CETP transfers neutral lipids from HDL to very low-density lipoprotein (VLDL) and is present in normal human plasma and serum. CETP is an important modulator of HDL cholesterol in humans and thus considered to be a therapeutic target for preventing cardiovascular diseases. The CETP Drug Screening Kit uses a donor molecule containing a fluorescent self-quenched neutral lipid that is transferred to an acceptor molecule in the presence of CETP (rabbit serum). CETP-mediated transfer of the fluorescent neutral lipid to the acceptor molecule results in an increase in fluorescence (Excitation: 465 nm; Emission: 535 nm). Inhibitor of CETP will inhibit the lipid transfer and therefore decrease fluorescence intensity. Amla extract had moderate CETP inhibitory activity. It showed 30% inhibition of CETP at lmg/ml.

With excellent anti-oxidant potentials discussed in Table A, it is also justified that continuous use of anti-oxidant medicaments containing standardized Emblica officinalis extracts enriched in greater than 40% w/w gallic acid esters obtained through the improvements of the present invention would help prevent the oxidation of low density lipoproteins and very low density lipoproteins in the serum. [Atherosclerosis: A Lesson in Lesions-Vadim Ivanov, Ph.D. LPI Cardiovascular Research Group]. With a moderate CETP inhibitory activity at 30% inhibition of CETP at a concentration of 1 mg/ml, it may be justified to conclude the potential of the standardized Emblica officinalis extract enriched in greater than 40% gallic acid esters obtained though improvements of the present invention for preventing the oxidation of low density lipoproteins and very low density lipoproteins in the serum.

TABLE A
ANTI-OXIDANT ACTIVITY

| ORAC | Standardized | Gallic acid | Vitamin C |
|---|---|---|---|
| (μmol TE/gm) | Emblica officinalis | | |
| | Extract enriched in > | | |
| | than 40% w/w of | | |
| | gallic acid esters | | |
| | including 1-O- | | |
| | galloyl-β-D-glucose | | |
| | and other mucic | | |
| | acid gallates | | |
| | obtained through | | |
| | improvements of | | |
| | the present | | |
| | invention. | | |
| | 2862 | 6712 | 3400 |
| DPPH IC50 (μg/ml) | 1.7 | 0.32 | 1.93 |

### EXAMPLE III

### Anti-5-Alpha-Reductase Activity (Table B. FIG. IV)

Table B shows the % 5-alpha reductase inhibition by the standardized Extract of amla enriched in greater than 40% w/w of gallic acid esters including 1-O-galloyl-β-D-glucose and other mucic acid gallates. With close to about 80% inhibition at a concentration of 250 ug/ml, it may be justified to conclude the potential of the standardized extract containing greater than 40% gallic acid esters obtained though improvements of the invention for the manufacture of medicaments to therapeutically manage of androgenic alopecia, chronic abacterial prostatitis, benign prostatic hyperplasia and prostatic cancer

TABLE B
5-ALPHA REDUCTASE ACTIVITY [% Inhibition]
Standardized Emblica officinalis extract enriched in > than 40% w/w of
gallic acid esters including 1-O-galloyl-β-D-glucose and other mucic acid
gallates obtained through improvements of the present invention.

| CONCENTRATIONS (ug/ml) | [% Inhibition] |
|---|---|
| 15.625 | 0 |
| 31.25 | 17.12 |
| 62.5 | 40.0 |
| 125 | 63.93 |
| 250 | 79.3 |

### EXAMPLE IV

### Anti-Diabetic Activity (Table C)

The anti-diabetic activity of the Emblica officinalis Extract enriched in >than 40% w/w of gallic acid esters including 1-O-galloyl-β-D-glucose and other mucic acid gallates obtained through improvements of the present invention. are discussed in Table C.
(i) Inhibition of Alpha Glucosidase: Alpha-glucosidase inhibitors are oral anti-diabetic drugs used for diabetes mellitus type 2 that work by preventing the digestion of carbohydrates (such as starch and table sugar). Carbohydrates are normally converted into simple sugars (monosaccharides) which can be absorbed through the intestine. Hence, alpha-glucosidase inhibitors reduce the impact of carbohydrates on blood sugar. Alpha-glucosidase inhibitors are saccharides that act as competitive inhibitors of enzymes needed to digest carbohydrates: specifically alpha-glucosidase enzymes in the brush border of the small intestines. The membrane-bound intestinal alpha-glucosidases hydrolyze oligosaccharides, trisaccharides, and disaccharides to glucose and other monosaccharides in the small intestine. Alpha-glucosidase inhibitors are used to establish greater glycemic control over hyperglycemia in diabetes mellitus type 2, particularly with regard to postprandial hyperglycemia. Acarbose which is a significant alpha glucosidase inhibitor reduces the post prandial blood sugar levels. Inhibition of these enzyme systems reduces the rate of digestion of carbohydrates. Less glucose is absorbed because the carbohydrates are not broken down into glucose molecules. In diabetic patients, the short-term effect of these drugs therapies is to decrease current blood glucose levels: the long term effect is a small reduction in hemoglobin. The assay for inhibitory activity is based on the principle that alpha glucosidase acts on substrate P-nitrophenyl α D-glucoside to form yellow colored P-nitrophenol and glucose. P-nitrophenol has an absorbance at 405 nm and is directly proportional to the enzyme activity. On treatment with the inhibitors, the absorbance due to P-nitrophenol is quenched as the enzyme activity is inhibited. The standardized Emblica officinalis extract enriched in >than 40% w/w of gallic acid esters including 1-O-galloyl-β-D-glucose and other mucic acid gallates obtained through improvements of the present invention showed 50% inhibition of Alpha glucosidase at 0.5 mg/ml.
(ii) DPP-IV Inhibition: DPP-IV inhibitors work by slowing the action of the enzyme responsible for glucagon-like-peptide-1 (GLP-1) breakdown. GLP-1 is a naturally occurring hormone that is produced and released by the gastrointestinal system. This hormone has several actions (Sharon H. Limaye Center for Diabetes Technology Assessment and Patient Education) including:
   1. Stimulates insulin production in the presence of hyperglycemia by delaying the breakdown of GLP-1
   2. Blocks the effects of glucagon (a hormone produced in the pancreas that signals the liver to release stored glucose into the bloodstream)
   3. Reduces appetite by delaying food absorption in the stomach potentially leading to weight loss
      DPP-IV inhibitors aimed at achieving the following:
1. Normal blood glucose levels
2. Reduction or elimination of the risk of hypoglycemia
3. Maintaining the pancreas' insulin-production capacity
4. Preventing weight gain associated with improved glucose control
5. Delaying the progression of type 2 diabetes and its secondary complications.
   DPP-IV inhibitory assay is based on the principle that DPP-IV acts on substrate peptide complex of Amino methyl coumarine (AMC) to form the cleaved fluorescent AMC. On treatment with the inhibitors, the fluorescence due to AMC is quenched as the enzyme activity is inhibited. The standardized Emblica officinalis extract enriched in >than 40% w/w of gallic acid esters including 1-O-galloyl-β-D-glucose and other mucic acid gallates obtained through improvements of the present invention show an IC₅₀ value of 750 μg/ml for DPP-IV inhibition.
(iii) Aldose Reductase Inhibition: The assay is based on the principle that Diabetic mellitus is a leading cause of many complications such as atherosclerosis,cardiac dysfunction, retinopathy and nephropathy. Cataract, the leading cause of blindness worldwide, is associated with many risk factors, a major one being diabetes. It is generally accepted that the polyol pathway of glucose metabolism plays a role in the development of cataract. Aldose reductase (AR) is the rate-limiting enzyme of this pathway. Aldose reductase, an NADPH dependent oxidoreductase, catalyses the reduction of glucose to the corresponding sugar alcohol, sorbitol. Sorbitol is subsequently metabolized to fructose by sorbitol dehydrogenase, the second enzyme in the pathway. The conversion of glucose to fructose by this means constitutes the polyol pathway of glucose metabolism. Aldose reductase acts on substrate DL glyceraldehyde which is oxidized in the presence of co factor NADPH to alcohol. This oxidation is inhibited in the presence of inhibitor. The aldose reductase inhibition (IC₅₀ ug/ml) values of 53.7 μg/ml as shown in Table C is much superior to the reported IC50 values 0.72 and 0.88 mg/ml respectively of the aqueous Emblica officinalis extract towards aldose reductase enzyme from rat lens and recombinant human aldose reductase[Mol Vis. Mar. 12, 2004;10:148-54]. With an enhanced antioxidant effect and inhibition of aldose reductase as shown herein above, Emblica officinalis extract enriched in >than 40% w/w of gallic acid esters including 1-O-galloyl-β-D-glucose and other mucic acid gallates obtained through improvements of the present invention. finds tremendous potential in the manufacture of medicaments for diabetic retinopathy and type II diabetes mellitus in general.

TABLE C
ANTI-DIABETIC ACTIVITY
Standardized Extract of amla containing >40% w/w
of gallic acid esters including 1-O-
galloyl-β-D-glucose and other mucic acid
gallates obtained through improvements of
the present invention.

| | | |
|---|---|---|
| Aldose reductase | 53.7 | μg/ml |
| inhibition (IC50) | | |
| ug/ml | | |
| DPPH IV | 750 | μg/ml |
| inhibition (IC50) | | |
| ug/ml | | |
| Alpha glucosidase | 0.5 | mg/ml |
| (IC50) ug/ml | | |

### EXAMPLE V

### Photoprotection to UVA and UVB (Table D)

The anti-UVA and anti-UVB activities (photoprotective activities) of the standardized Extract of amla containing >40% w/w of gallic acid esters including 1-O-galloyl-β-D-glucose and other mucic acid gallates obtained through the improvements of the present invention are discussed in Table D. While US20050089590 discusses Metal Chelation Ability as an index of anti-oxidant activity towards oxidative damage caused by rays of the sun, thereby attempting to prove the photo protective activity of a standardized extract of Emblica officinalis containing Emblicanin A, Emblicanin B, Pendunculagin and Punigluconin, the present inventors who have confirmed the absence of these molecules in their extract of Emblica officinalis which is enriched in greater than 40% w/w of gallic acid esters including 1-O-galloyl-β-D-glucose and other mucic acid gallates obtained through improvements of the present invention, demonstrate the potential of said gallic acid ester enriched extract towards photoprotection in in vitro cell based assays discussed herein below.
(i) Melanin inhibition in B16F1 mouse melanoma cell line—Hyperpigmentation is a process of darkening of skin due to uncontrolled melanogenesis activity in the melanocyte cells of the skin. Hence, inhibition of melanin production helps in lightening the skin tone. Melanin inhibition in B16F1 mouse melanoma cell line is studied by treating MSH induced cells for a period of 9 days after which the melanin is extracted by 1N NaOH and quantitated.
(ii) UV protection: Varying concentrations of the test sample in the cell culture medium are added to the 24 hr monolayers of cells of Swiss 3T3 mouse fibroblast cells in a 96 well plate and then exposed to UV A and UV B irradiation dose. Each concentration was applied with ‘n’ value of 12. The plate layout was made in such a way that six of the replicates were exposed to UV and six unexposed. The unexposed region of the plate was covered by aluminum foil. After exposure, the cells were incubated in a CO₂ incubator for 48 hrs and developed by the NRU staining techniques to analyze the cell viability. The percentage of UV protection was calculated with respect to the cytotoxicity in exposed cells as compared to that of the unexposed cells.

TABLE D
PHOTOPROTECTIVE ACTIVITY AGAINST UVA and UVB RAYS
Standardized Emblica officinalis Extract enriched with > than
40% w/w of gallic acid esters including 1-O-galloyl-β-D-glucose
and other mucic acid gallates obtained through improvements
of the present invention.

| | | |
|---|---|---|
| Melanin Inhibition in B16 F1 Cells IC50 (μg/ml) | 12 | μg/ml |
| UV B protection in Swiss 3T3 Cells EC50 (μg/ml) | 41.2 | μg/ml |
| (effective concentration for 50% UV protection) | | |
| UV A protection in Swiss 3T3 Cells EC50 (μg/ml) | 14 | μg/ml |
| (effective concentration for 50% UV protection) | | |

With activity towards both UVA and UVB rays as highlighted in Table D and excellent anti-oxidant potential as highlighted in Table A, it is well justified that the standardized extract of amla containing >40% w/w of gallic acid esters including 1-O-galloyl-β-D-glucose and other mucic acid gallates obtained through improvements of the present invention have tremendous potential for use in the manufacture of medicaments to retard, prevent and reverse undesirable changes in the skin caused by exposure to UVA and UVB rays.

With reference to the aforementioned examples the present invention is marked by the following preferred embodiments also.

A stabilized anti-oxidant formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained through improvement in the method of enriching said gallic acid esters by including Super Critical Fluid Extraction (SCFE) process after the steps of the aqueous extraction of the fruits of Emblica officinalis, SCFE selectively removing medium polar constituents including gallic acid represented by STR#6 and ellagic acid represented by STR#7 from the aqueous extract to result in a standardized Emblica officinalis extract enriched in the said gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

A stabilized anti-oxidant formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained by the improvement consisting essentially the use of Super Critical Fluid Extraction (SCFE) process as the principal extraction process to obtain standardized enriched gallic acid esters from the fruits of Emblica officinalis by overcoming the disadvantages of conventional extraction methods where hydrolysis of the gallic acid esters to gallic acid represented by STR#6 yield low levels of gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. Still more preferably, the entrant is acetone or an alcohol selected from the group consisting of aliphatic and aromatic alcohols.

A stabilized anti-UVA and anti-UVB formulation (photo protective formulation) from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained through improvement in the method of enriching said gallic acid esters by including Super Critical Fluid Extraction (SCFE) process after the steps of the aqueous extraction of the fruits of Emblica officinalis, SCFE selectively removing medium polar constituents including gallic acid represented by STR#6 and ellagic acid represented by STR#7 from the aqueous extract to result in a standardized Emblica officinalis extract enriched in the said gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

A stabilized anti-UVA and anti-UVB formulation (photo protective formulation) from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained by the improvement consisting essentially the use of Super Critical Fluid Extraction (SCFE) process as the principal extraction process to obtain standardized enriched gallic acid esters from the fruits of Emblica officinalis by overcoming the disadvantages of conventional extraction methods where hydrolysis of the gallic acid esters to gallic acid represented by STR#6 yield low levels of gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. Still more preferably, the entrant is acetone or an alcohol selected from the group consisting of aliphatic and aromatic alcohols.

A stabilized melanogenesis inhibitory formulation (photo protective formulation) from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained through improvement in the method of enriching said gallic acid esters by including Super Critical Fluid Extraction (SCFE) process after the steps of the aqueous extraction of the fruits of Emblica officinalis, SCFE selectively removing medium polar constituents including gallic acid represented by STR#6 and ellagic acid represented by STR#7 from the aqueous extract to result in a standardized Emblica officinalis extract enriched in the said gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

A stabilized melanogenesis inhibitory formulation (photo protective formulation) from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained by the improvement consisting essentially the use of Super Critical Fluid Extraction (SCFE) process as the principal extraction process to obtain standardized enriched gallic acid esters from the fruits of Emblica officinalis by overcoming the disadvantages of conventional extraction methods where hydrolysis of the gallic acid esters to gallic acid represented by STR#6 yield low levels of gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. Still more preferably, the entrant is acetone or an alcohol selected from the group consisting of aliphatic and aromatic alcohols.

A stabilized 5-alpha-reductase inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained through improvement in the method of enriching said gallic acid esters by including Super Critical Fluid Extraction (SCFE) process after the steps of the aqueous extraction of the fruits of Emblica officinalis, SCFE selectively removing medium polar constituents including gallic acid represented by STR#6 and ellagic acid represented by STR#7 from the aqueous extract to result in a standardized Emblica officinalis extract enriched in the said gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

A stabilized 5-alpha-reductase inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained by the improvement consisting essentially the use of Super Critical Fluid Extraction (SCFE) process as the principal extraction process to obtain standardized enriched gallic acid esters from the fruits of Emblica officinalis by overcoming the disadvantages of conventional extraction methods where hydrolysis of the gallic acid esters to gallic acid represented by STR#6 yield low levels of gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. Still more preferably, the entrant is acetone or an alcohol selected from the group consisting of aliphatic and aromatic alcohols.

A stabilized DPP-IV inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained through improvement in the method of enriching said gallic acid esters by including Super Critical Fluid Extraction (SCFE) process after the steps of the aqueous extraction of the fruits of Emblica officinalis, SCFE selectively removing medium polar constituents including gallic acid represented by STR#6 and ellagic acid represented by STR#7 from the aqueous extract to result in a standardized Emblica officinalis extract enriched in the said gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

A stabilized DPP-IV inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained by the improvement consisting essentially the use of Super Critical Fluid Extraction (SCFE) process as the principal extraction process to obtain standardized enriched gallic acid esters from the fruits of Emblica officinalis by overcoming the disadvantages of conventional extraction methods where hydrolysis of the gallic acid esters to gallic acid represented by STR#6 yield low levels of gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. Still more preferably, the entrant is acetone or an alcohol selected from the group consisting of aliphatic and aromatic alcohols.

A stabilized aldose reductase inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained through improvement in the method of enriching said gallic acid esters by including Super Critical Fluid Extraction (SCFE) process after the steps of the aqueous extraction of the fruits of Emblica officinalis, SCFE selectively removing medium polar constituents including gallic acid represented by STR#6 and ellagic acid represented by STR#7 from the aqueous extract to result in a standardized Emblica officinalis extract enriched in the said gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

A stabilized aldose reductase inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained by the improvement consisting essentially the use of Super Critical Fluid Extraction (SCFE) process as the principal extraction process to obtain standardized enriched gallic acid esters from the fruits of Emblica officinalis by overcoming the disadvantages of conventional extraction methods where hydrolysis of the gallic acid esters to gallic acid represented by STR#6 yield low levels of gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. Still more preferably, the entrant is acetone or an alcohol selected from the group consisting of aliphatic and aromatic alcohols.

A stabilized alpha glucosidase inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained through improvement in the method of enriching said gallic acid esters by including Super Critical Fluid Extraction (SCFE) process after the steps of the aqueous extraction of the fruits of Emblica officinalis, SCFE selectively removing medium polar constituents including gallic acid represented by STR#6 and ellagic acid represented by STR#7 from the aqueous extract to result in a standardized Emblica officinalis extract enriched in the said gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

A stabilized alpha glucosidase inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained by the improvement consisting essentially the use of Super Critical Fluid Extraction (SCFE) process as the principal extraction process to obtain standardized enriched gallic acid esters from the fruits of Emblica officinalis by overcoming the disadvantages of conventional extraction methods where hydrolysis of the gallic acid esters to gallic acid represented by STR#6 yield low levels of gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. Still more preferably, the entrant is acetone or an alcohol selected from the group consisting of aliphatic and aromatic alcohols.

A stabilized Cholesteryl ester transfer protein inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained through improvement in the method of enriching said gallic acid esters by including Super Critical Fluid Extraction (SCFE) process after the steps of the aqueous extraction of the fruits of Emblica officinalis, SCFE selectively removing medium polar constituents including gallic acid represented by STR#6 and ellagic acid represented by STR#7 from the aqueous extract to result in a standardized Emblica officinalis extract enriched in the said gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting the spray dried aqueous extract of the fresh fruits of Emblica officinalis loaded to a SCFE extractor, using a pump speed of 25 kg/hr at 100 to 120 bar pressure for 1 hour, followed by 120 to 150 bar pressure for 1 hour, followed by 175 to 200 bar pressure for 1 hour and then 300 to 350 bar pressure for 3.5 hours with the temperature continuously maintained at 50° C. during the said extraction process.

A stabilized Cholesteryl ester transfer protein inhibitory formulation from Emblica officinalis enriched in greater than 40% w/w of gallic acid esters including 1-O-galloyl-β-D-glucose represented by STR#1 and and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5, said formulation obtained by the improvement consisting essentially the use of Super Critical Fluid Extraction (SCFE) process as the principal extraction process to obtain standardized enriched gallic acid esters from the fruits of Emblica officinalis by overcoming the disadvantages of conventional extraction methods where hydrolysis of the gallic acid esters to gallic acid represented by STR#6 yield low levels of gallic acid esters. Further, the said formulation is devoid of (i) 2,3-di-O-galloyl-4,6-(S)-hexahydroxydiphenoyl-2-keto-glucono-lactone; (ii) 2,3,4,6-bis-(S)-hexahydroxydiphenoyl-2-keto-glucono-lactone; (iii) 2,3-di-O-galloyl4,6-(S)-hexahydroxydiphenoyl gluconic acid; (iv) 2,3,4,6-bis-(S)-hexahydroxydiphenoyl-D-glucose; and (v) Ascorbic acid.

More preferably, the Super Critical Fluid Extraction (SCFE) process discussed in the previous paragraph comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C. Still more preferably, the entrant is acetone or an alcohol selected from the group consisting of aliphatic and aromatic alcohols.

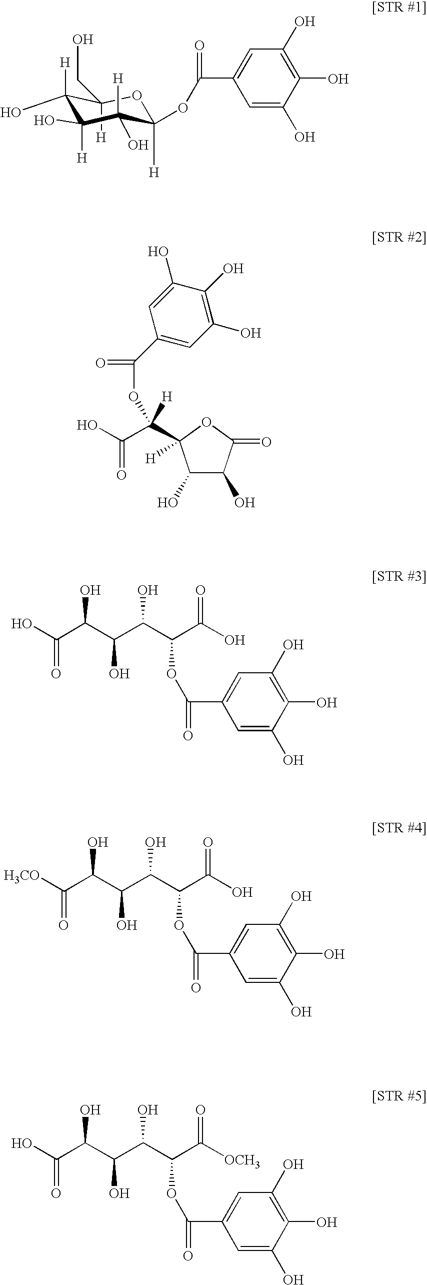

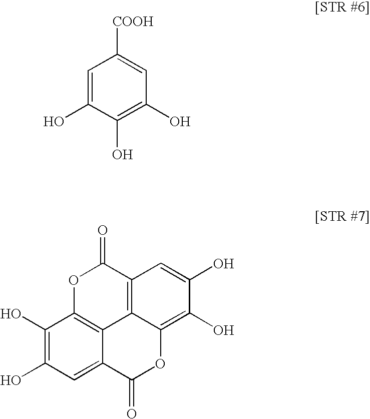

The advantages of the present invention include novel improvements in methods to enrich the essential components in the fruits of Emblica officinalis namely gallic acid esters which include 1-O-galloyl-β-D-glucose and other mucic acid gallates (enrichment greater than 40% w/w of gallic acid esters). The invention has also disclosed novel, enhanced biological properties of the said enriched gallic acid esters, uses thereof as medicaments and methods of treatment also.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. In the art for the aqueous extraction of gallic acid esters including 1-O-galloyl-β-D-glucose represented by STR#1 and mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 from the fruits of Emblica officinalis, an improvement in the method of enriching said gallic acid esters by including Super Critical Fluid Extraction (SCFE) process after the steps of the aqueous extraction wherein SCFE selectively removes medium polar constituents including gallic acid represented by STR#6 and ellagic acid represented by STR#7 from the aqueous extract to result in a standardized Emblica officinalis extract enriched in the said gallic acid esters.

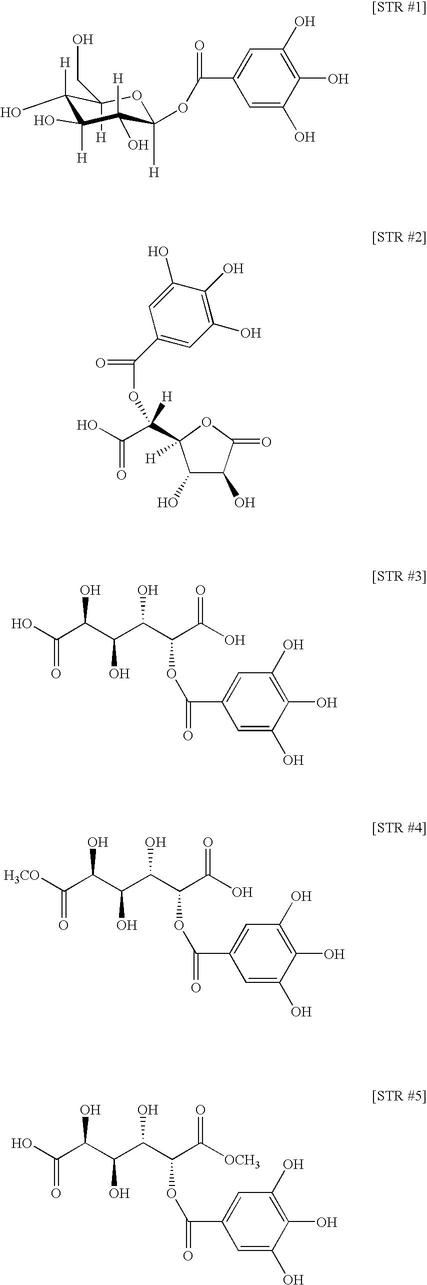

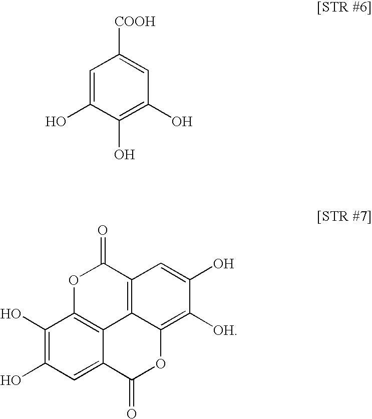

2. The improvement according to claim 1, wherein the Super Critical Fluid Extraction (SCFE) process comprises the step of extracting the spray dried aqueous extract of the fresh fruits of Emblica officinalis loaded to a SCFE extractor using a pump speed of 25 kg/hr at 100 to 120 bar pressure for 1 hour, followed by 120 to 150 bar pressure for 1 hour, followed by 175 to 200 bar pressure for 1 hour and then 300 to bar pressure for 3.5 hours with the temperature continuously maintained at 50° C. during the said extraction process.

3. In the art for the extraction of gallic acid esters including 1-O-galloyl-β-D-glucose represented by STR#1 and mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 from Emblica officinalis, the improvement consisting essentially the use of Super Critical Fluid Extraction (SCFE) process as the principal extraction process to obtain standardized enriched gallic acid esters by overcoming the disadvantages of conventional extraction methods where hydrolysis of the gallic acid esters to gallic acid represented by STR#6 yield low levels of gallic acid esters.

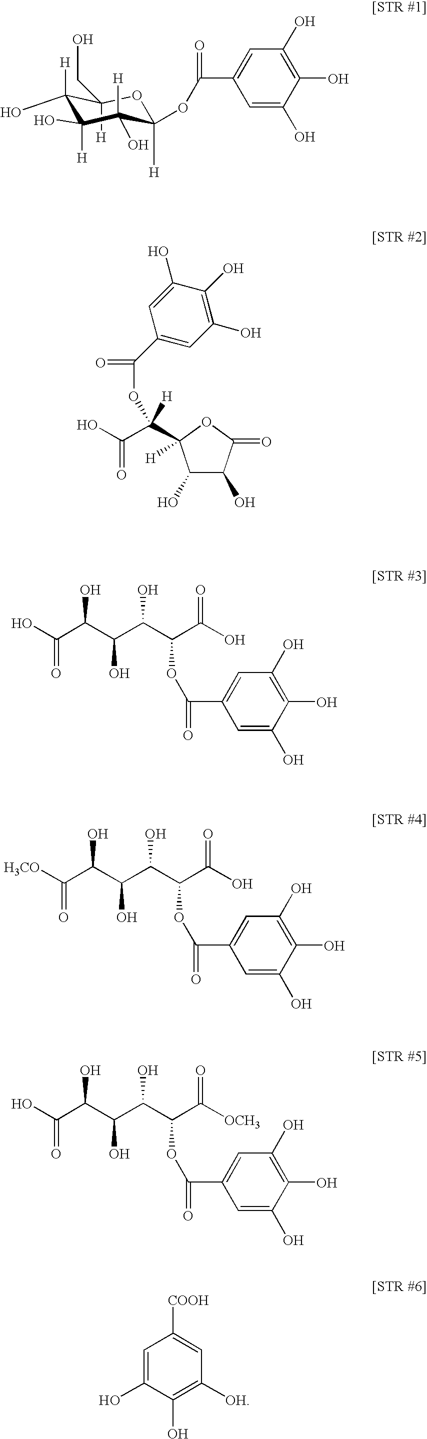

4. The improvement according to claim 3, wherein the Super Critical Fluid Extraction (SCFE) process comprises the step of extracting fresh cut fruits of Emblica officinalis loaded on to a Supercritical fluid extractor using 200 ml of an entrant at a pump speed of 21 kg/hr at 150 to 200 bar pressure for 1 hour, followed by 300 to 350 bar pressure for 3 hours, the entire extraction process carried out at a temperature of 45° C.

5. The improvement according to claim 4, wherein the entrant is acetone.

6. The improvement according to claim 4, wherein the entrant is an alcohol.

7. The improvements as in claims 1 and 3, in which the standardized Emblica officinalis extract is enriched in greater than 40% w/w of gallic acid esters.

8. An extract of Emblica officinalis produced by the improvements as in one of claims 1 and 3.

9. The improvement as in one of claims 1 and 3, in which the standardized Emblica officinalis extracts enriched in greater than 40% w/w of gallic acid esters are devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose;
and
5. Ascorbic acid.

10. Pharmaceutical compositions comprising standardized Emblica officinalis extracts enriched in greater than 40% w/w of gallic acid esters through the improvements as in one of claims 1 and 3, in which said extracts are devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose;
and
5. Ascorbic acid.

11. Nutraceutical compositions comprising standardized Emblica officinalis extracts enriched in greater than 40% w/w of gallic acid esters through the improvements as in one of claims 1 and 3, in which said extracts are devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose;
and
5. Ascorbic acid.

12. Cosmetic compositions comprising standardized Emblica officinalis extracts enriched in greater than 40% w/w of gallic acid esters through the improvements as in one of claims 1 and 3, in which said extracts are devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose;
and
5. Ascorbic acid.

13. The use of standardized Emblica officinalis extracts enriched in greater than 40% w/w of gallic acid esters through the improvements as in one of claims 1 and 3 in the manufacture of medicaments for the treatment of androgenic alopecia.

14. The use of standardized Emblica officinalis extracts enriched in greater than 40% w/w of gallic acid esters through the improvements as in one of claims 1 and 3 in the manufacture of medicaments for the treatment of chronic abacterial prostatitis/chronic pelvic pain syndrome.

15. The use of standardized Emblica officinalis extracts enriched in greater than 40% w/w of gallic acid esters through the improvements as in one of claims 1 and 3 in the manufacture of medicaments for the treatment of benign prostatic hyperplasia.

16. The use of standardized Emblica officinalis extracts enriched in greater than 40% w/w of gallic acid esters through the improvements as in one of claims 1 and 3 in the manufacture of medicaments for the treatment of prostatic cancer.

17. The use of standardized Emblica officinalis extracts enriched in greater than 40% w/w of gallic acid esters through the improvements as in one of claims 1 and 3 in the manufacture of cosmetic medicaments for skin protection against UVA and UVB rays.

18. The use of standardized Emblica officinalis extracts enriched in greater than 40% w/w of gallic acid esters through the improvements as in one of claims 1 and 3 in the manufacture of medicaments for the treatment of diabetic retinopathy.

19. The use of standardized Emblica officinalis extracts enriched in greater than 40% w/w of gallic acid esters through the improvements as in one of claims 1 and 3 in the manufacture of medicaments for the treatment of Type II diabetes mellitus.

20. The use of standardized Emblica officinalis extracts enriched in greater than 40% w/w of gallic acid esters through the improvements as in one of claims 1 and 3 in the manufacture of anti-oxidant medicaments for preventing the oxidation of serum low density and very low density lipoproteins.

21. The standardized extracts as in one of claims 13, 14, 15, 16, 17, 18, 19 and 20 which are devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose;
and
5. Ascorbic acid.

22. The methods of treatment as in one of claims 13, 14, 15, 16, 17, 18, 19 and 20 comprising the oral administration of an effective amount of 400 mg/day of standardized Emblica officinalis extracts enriched in greater than 40% w/w of gallic acid esters through the improvements as in claims 1 and 3 in patients who are in need of such treatment.

23. The method of treatment according to claim 18, wherein topical medicaments comprising standardized Emblica officinalis extracts enriched in greater than 40% w/w of gallic acid esters through the improvements as in claims 1 and 3, are applied in exposed areas of skin at a risk for UVA and UVB damage.

24. A stabilized anti-oxidant formulation obtained from the fruits of Emblica officinalis, enriched in greater than 40% w/w of gallic acid esters including 1-O-galloyl-β-D-glucose represented by STR#1 and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 through the improvements as in one of claims 1 and 3, wherein said formulation is devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose; and
5. Ascorbic acid.

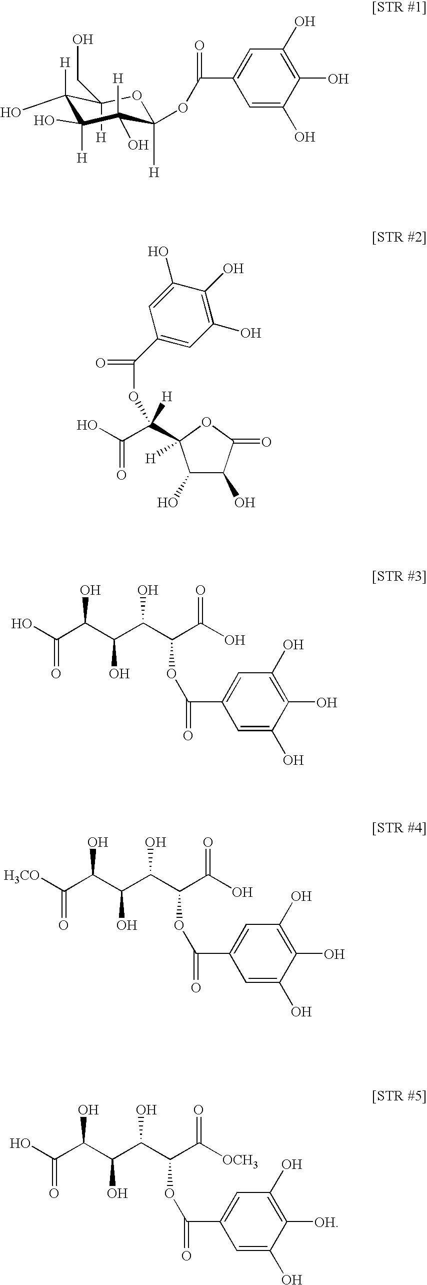

25. A stabilized anti-UVA and anti-UVB formulation (photo protective formulation) obtained from the fruits of Emblica officinalis, enriched in greater than 40% w/w of gallic acid esters including 1-O-galloyl-β-D-glucose represented by STR#1 and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 through the improvements as in one of claims 1 and 3, wherein said formulation is devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose; and
5. Ascorbic acid.

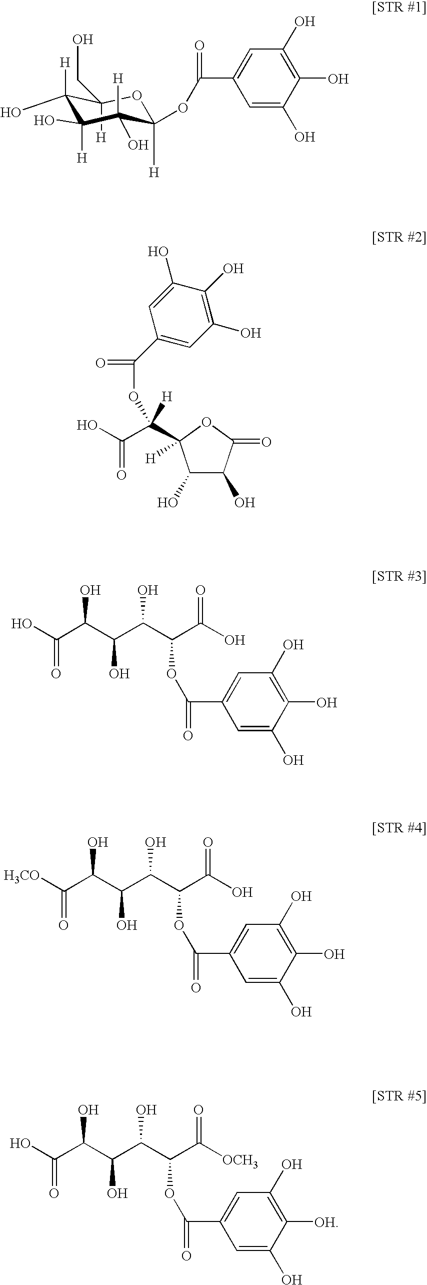

26. A stabilized melanogenesis inhibitory formulation (photo protective formulation) obtained from the fruits of Emblica officinalis, enriched with greater than 40% w/w of gallic acid esters including 1-O-galloyl-β-D-glucose represented by STR#1 and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 through the improvements as in one of claims 1 and 3, wherein said formulation is devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose; and
5. Ascorbic acid.

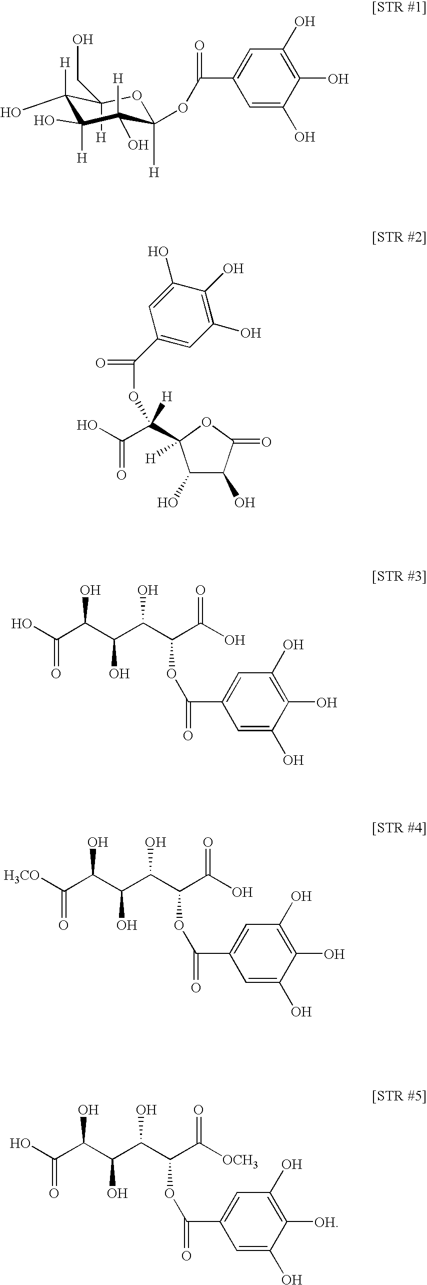

27. A stabilized 5-alpha-reductase inhibitory formulation obtained from the fruits of Emblica officinalis, enriched in greater than 40% w/w of gallic acid esters including 1-O-galloyl-β-D-glucose represented by STR#1 and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 through the improvements as in one of claims 1 and 3, wherein said formulation is devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose; and
5. Ascorbic acid.

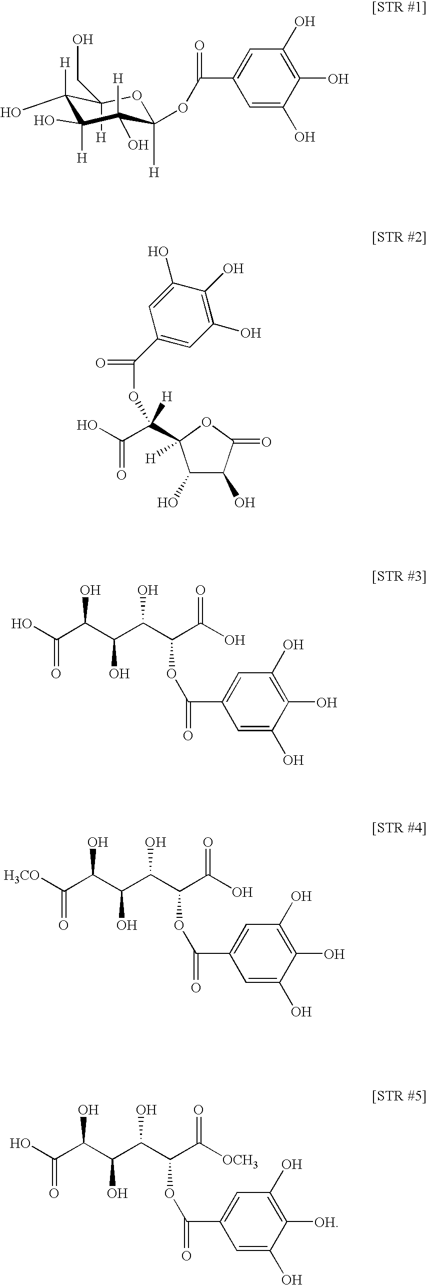

28. A stabilized alpha glucosidase inhibitory formulation from the fruits of Emblica officinalis enriched in greater than 40% w/w of gallic acid esters including 1-O-galloyl-β-D-glucose represented by STR#1 and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 through the improvements as in one of claims 1 and 3, wherein said formulation is devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose; and
5. Ascorbic acid.

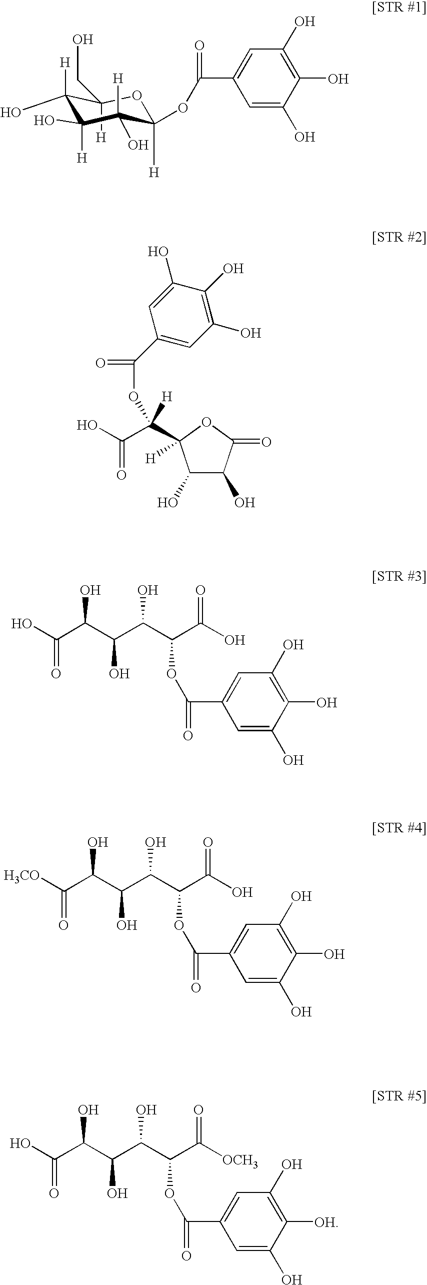

29. A stabilized aldose reductase inhibitory formulation obtained from the fruits of Emblica officinalis, enriched in greater than 40% w/w of gallic acid esters including 1-O-galloyl-β-D-glucose represented by STR#1 and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 through the improvements as in one of claims 1 and 3, wherein said formulation is devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose; and
5. Ascorbic acid.

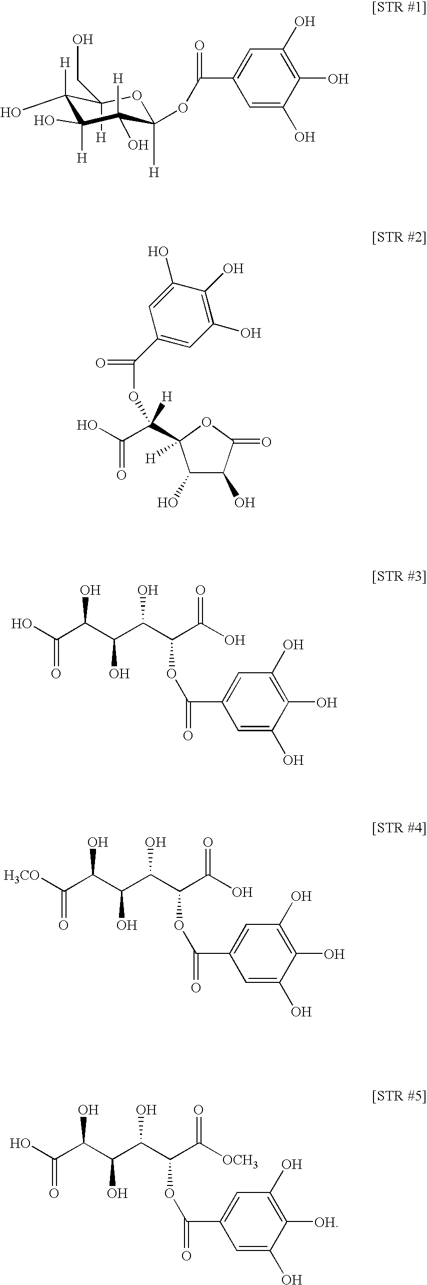

30. A stabilized dipeptidyl aminopeptidase IV (DPP IV) inhibitory formulation obtained from the fruits of Emblica officinalis, enriched in greater than 40% w/w of gallic acid esters including 1-O-galloyl-β-D-glucose represented by STR#1 and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 through the improvements as in one of claims 1 and 3, wherein said formulation is devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose; and
5. Ascorbic acid.

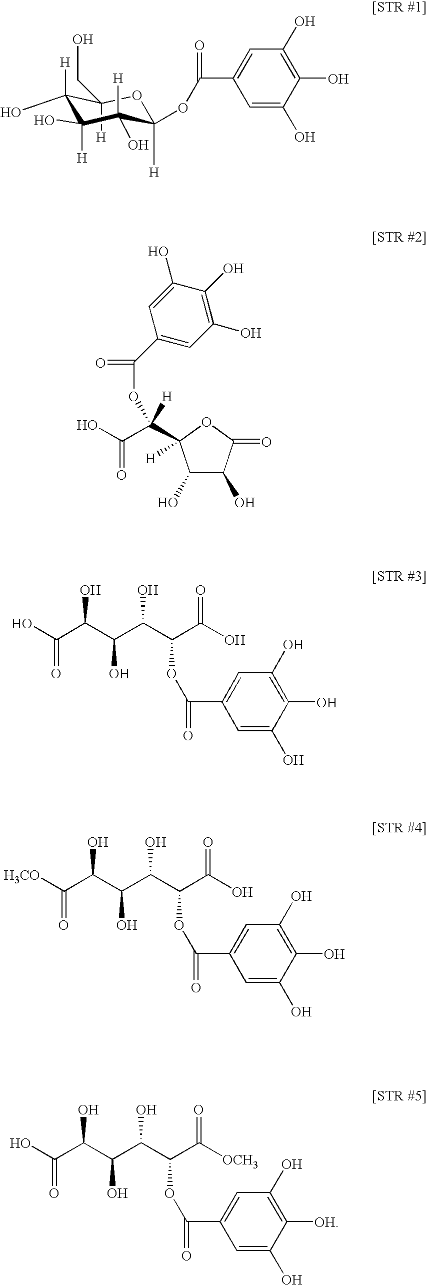

31. A stabilized Cholesteryl ester transfer protein (CETP) inhibitory formulation obtained from the fruits of Emblica officinalis, enriched in greater than 40% w/w of gallic acid esters including 1-O-galloyl-β-D-glucose represented by STR#1 and other mucic acid gallates namely mucic acid 1,4-lactone 5-O-gallate represented by STR#2, mucic acid 2-O-gallate represented by STR#3, mucic acid 6-Methyl ester 2-O-gallate represented by STR#4 and mucic acid 1-Methyl ester 2-O-gallate represented by STR#5 through the improvements as in one of claims 1 and 3, wherein said formulation is devoid of
1. 2,3-di-O-galloyl-4,6-(S)-hexahydroxydiphenoyl-2-keto-glucono-lactone;
2. 2,3,4,6-bis-(S)-hexahydroxydiphenoyl-2-keto-glucono-lactone;
3. 2,3-di-O-galloyl4,6-(S)-hexahydroxydiphenoyl gluconic acid;
4. 2,3,4,6-bis-(S)-hexahydroxydiphenoyl-D-glucose; and
5. Ascorbic acid. [chemical structure]
